SEQUENCE LISTING (5 sequences)

SEQ ID NO: 1 - protein, 732 aa, Homo sapiens
MVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISLQFFLPFSLGKEDGSGDRGDGPFRLGGHGPSFPLKGITEQQKEGLEIVKMVMISLEGEDGLDEIYSFSESLRKLCVFKKIERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAKKLKTEQGGAHFSVSSLAEGSVTSVGSVNPAENFRVLVKQKKASFEEASNQLINHIEQFLDTNETPYFMKSIDCIRAFREEAIKFSEEQRFNNFLKALQEKVEIKQLNHFWEIVVQDGITLITKEEASGSSVTAEEAKKFLAPKDKPSGDTAAVFEEGGDVDDLLDMI

SEQ ID NO: 2 - DNA, 3304 bp, Homo sapiens
cgaccaaagcgcctgaggaccggcaacatggtgcggtcggggaataaggcagctgttgtgctgtgtatggacgtgggctttaccatgagtaactccattcctggtatagaatccccatttgaacaagcaaagaaggtgataaccatgtttgtacagcgacaggtgtttgctgagaacaaggatgagattgctttagtcctgtttggtacagatggcactgacaatcccctttctggtggggatcagtatcagaacatcacagtgcacagacatctgatgctaccagattttgatttgctggaggacattgaaagcaaaatccaaccaggttctcaacaggctgacttcctggatgcactaatcgtgagcatggatgtgattcaacatgaaacaataggaaagaagtttgagaagaggcatattgaaatattcactgacctcagcagccgattcagcaaaagtcagctggatattataattcatagcttgaagaaatgtgacatctccctgcaattcttcttgcctttctcacttggcaaggaagatggaagtggggacagaggagatggcccctttcgcttaggtggccatgggccttcctttccactaaaaggaattaccgaacagcaaaaagaaggtcttgagatagtgaaaatggtgatgatatctttagaaggtgaagatgggttggatgaaatttattcattcagtgagagtctgagaaaactgtgcgtcttcaagaaaattgagaggcattccattcactggccctgccgactgaccattggctccaatttgtctataaggattgcagcctataaatcgattctacaggagagagttaaaaagacttggacagttgtggatgcaaaaaccctaaaaaaagaagatatacaaaaagaaacagtttattgcttaaatgatgatgatgaaactgaagttttaaaagaggatattattcaagggttccgctatggaagtgatatagttcctttctctaaagtggatgaggaacaaatgaaatataaatcggaggggaagtgcttctctgttttgggattttgtaaatcttctcaggttcagagaagattcttcatgggaaatcaagttctaaaggtctttgcagcaagagatgatgaggcagctgcagttgcactttcctccctgattcatgctttggatgacttagacatggtggccatagttcgatatgcttatgacaaaagagctaatcctcaagtcggcgtggcttttcctcatatcaagcataactatgagtgtttagtgtatgtgcagctgcctttcatggaagacttgcggcaatacatgttttcatccttgaaaaacagtaagaaatatgctcccaccgaggcacagttgaatgctgttgatgctttgattgactccatgagcttggcaaagaaagatgagaagacagacacccttgaagacttgtttccaaccaccaaaatcccaaatcctcgatttcagagattatttcagtgtctgctgcacagagctttacatccccgggagcctctacccccaattcagcagcatatttggaatatgctgaatcctcccgctgaggtgacaacaaaaagtcagattcctctctctaaaataaagaccctttttcctctgattgaagccaagaaaaaggatcaagtgactgctcaggaaattttccaagacaaccatgaagatggacctacagctaaaaaattaaagactgagcaagggggagcccacttcagcgtctccagtctggctgaaggcagtgtcacctctgttggaagtgtgaatcctgctgaaaacttccgtgttctagtgaaacagaagaaggccagctttgaggaagcgagtaaccagctcataaatcacatcgaacagtttttggatactaatgaaacaccgtattttatgaagagcatagactgcatccgagccttccgggaagaagccattaagttttcagaagagcagcgctttaacaacttcctgaaagcccttcaagagaaagtggaaattaaacaattaaatcatttctgggaaattgttgtccaggatggaattactctgatcaccaaagaggaagcctctggaagttctgtcacagctgaggaagccaaaaagtttctggcccccaaagacaaaccaagtggagacacagcagctgtatttgaagaaggtggtgatgtggacgatttattggacatgatataggtcgtggatgtatggggaatctaagagagctgccatcgctgtgatgctgggagttctaacaaaacaagttggatgcggccattcaaggggagccaaaatctcaagaaattcccagcaggttacctggaggcggatcatctaattctctgtggaatgaatacacacatatatattacaagggataatttagaccccatacaagtttataaagagtcattgttattttctggttggtgtattattttttctgtggtcttactgatctttgtatattacatacatgctttgaagtttctggaaagtagatcttttcttgacctagtatatcagtgacagttgcagcccttgtgatgtgattagtgtctcatgtggaaccatggcatggttattgatgagtttcttaaccctttccagagtcctcctttgcctgatcctccaacagctgtcacaacttgtgttgagcaagcagtagcatttgcttcctcccaacaagcagctgggttaggaaaaccatgggtaaggacggactcacttctctttttagttgaggccttctagttaccacattactctgcctctgtatataggtggttttctttaagtggggtgggaaggggagcacaatttcccttcatactccttttaagcagtgagttatggtggtggtctcatgaagaaaagaccttttggcccaatctctgccatatcagtgaacctttagaaactcaaaaactgagaaatttacttcagtagttagaattatatcacttcactgttctctacttgcaagcctcaaagagagaaagtttcgttatattaaaacacttaggtaacttttcggtctttcccatttctacctaagtcagctttcatctttgtggatggtgtctcctttactaaataagaaaataacaaagcccttattctctttttttcttgtcctcattcttgccttgagttccagttcctctttggtgtacagacttcttggtacccagtcacctctgtcttcagcaccctcataagtcgtcactaatacacagttttgtacatgtaacattaaaggcataaatgactc

SEQ ID NO: 3 - DNA, 23 bp, Artificial Sequence
  Oligonucleotide primer for real-time detection PCR
ccctagaaaacccatcctctgtg

SEQ ID NO: 4 - DNA, 23 bp, Artificial Sequence
  Oligonucleotide primer for real-time detection PCR
aggttctgcatgactgctactgg

SEQ ID NO: 5 - DNA, 27 bp, Artificial Sequence
  Oligonucleotide probe for real-time detection PCR
tcatggacagttatctgaccaccccca

(19) United States
(12) Patent Application Publication
(10) Pub. No.: US 2006/0188923 A1
(43) Pub. Date: Aug. 24, 2006

(54) Novel human parvovirus B19 receptor and uses thereof

(76) Inventors: Yasuhiko Munakata, Sendai-shi (JP); Takako Saito, Sendai-shi (JP)

Correspondence Address: BIRCH STEWART KOLASCH & BIRCH, PO BOX 747, FALLS CHURCH, VA, 22040-0747, US

(21) Appl. No.: 11/412,092

(22) Filed: Apr. 27, 2006

Related U.S. Application Data
(62) Division of application No. 10/768,030, filed on Feb. 2, 2004.

(30) Foreign Application Priority Data
Aug. 1, 2003 (JP) 2003-205279

(51) Int. Cl. C12Q 1/70; C12Q 1/68

(52) U.S. Cl. 435/6; 435/5

(57) ABSTRACT

A novel receptor for human parvovirus B19, other than P antigen, as well as a reagent for measuring human parvovirus B19, a reagent for adsorbing human parvovirus B19, and an agent for suppressing infection, which utilize the receptor for human parvovirus B19 is disclosed. It was discovered that Ku 80 protein is a receptor for human parvovirus B19. Thus, a receptor for human parvovirus B19 consisting essentially of Ku80 was provided. The agent for binding human parvovirus B19 comprises Ku80. The agent for suppressing infection by human parvovirus B19 comprises a substance which inhibits the binding between Ku80 and human parvovirus B19 or a binding fragment thereof.

This application is a Divisional of co-pending application Ser. No. 10/768,030 filed on Feb. 2, 2004, and for which priority is claimed under 35 U.S.C. § 120; and this application claims priority of Application No. 2003-205279 filed in Japan on Aug. 1, 2003 under 35 U.S.C. § 119; the entire contents of all are hereby incorporated by reference.

### TECHNICAL FIELD TO WHICH THE INVENTION PERTAINS

The present invention relates to a novel human parvovirus B19 receptor and uses thereof, as well as to a process for producing a cell which presents the same.

### PRIOR ART

Human parvovirus B19 (hereinafter also referred to as simply “B19” for short) is a single-stranded DNA virus which causes various diseases including erythema infectiosum of infants (Reference 1), hydrops fetalis caused when a pregnant woman is infected (Reference 2), acute pure red cell anemia (Reference 3) and multiple arthritis of adults (References 4 and 5). As an infection receptor of B19, P antigen (Globoside) which is a blood type glycoprotein expressed on the membranes of erythrocytes was identified in 1993 by Young et al. (Reference 6). This was also supported by the fact that in clinical cases which exhibit resistance to B19 infection, the phenotype lacking the expression of P antigen is presented (Reference 7). Thus, it has been understood that P antigen is the infection receptor of B19 and erythroblasts which highly express P antigen are the infection target cells. In B19 infectious diseases, anemia due to the infection to erythroblasts is the main symptom. However, there are some cases wherein symptoms such as leukopenia and thrombocytopenia (Reference 8), and phenomena showing immune disorder, such as emerging of autoantibody, are observed. Further, cases in which rheumatoid arthritis was caused after B19 infection were reported, and existence of B19 DNA in peripheral blood granulocyte or in joints was proved (References 9, 10 and 11). Thus, there are some cases which are difficult to explain based on the infection of B19 to erythroblasts alone. Thus, in B19 infectious diseases, it has been pointed out that there would be an unknown manner of B19 infection, other than the infection to erythroblasts through P antigen. On the other hand, based on the studies using B19 infection-sensitive cell lines, since no correlation is observed between the amount of the expressed P antigen and the infection efficiency of B19, it has been pointed out that a B19 infection-related molecule (co-receptor or the like) other than P antigen may exist (Reference 12). Thus, although the existence of a B19 infection-related molecule, other than P antigen is assumed, nothing has been identified so far. It has been reported that a plurality of molecules participate as an infection receptor for a virus, and they are involved in the virus infection as co-receptors. For example, it has been proved that a chemokine receptor functions as a co-receptor for human immunodeficiency virus (HIV) (Reference 13), very late antigen 2 (VLA2) functions as a co-receptor for echovirus (Reference 14), and αVβ5 integrin functions as a co-receptor for adeno associated virus 2 (AAV2) (Reference 15), and these molecules play important roles in determining sensitivity to virus infection and infection specificity. As for B19, like other viruses, the possibility that an infection receptor other than P antigen or a co-receptor exists is expected. To identify the molecule involved in B19 infection will attribute not only to the clarification of the mechanism of infection by B19, but also to the understanding of various symptoms associated with B19 infection, so that it may also be information useful for diagnoses and therapies of B19 infectious diseases.

The present inventors have discovered that B19-VP1 protein which is a structural protein of B19 in immunocytes such as T lymphocytes, B lymphocytes, dendrocytes and macrophages, which infiltrated into synovial membrane tissues of joints of patients suffering from rheumatoid arthritis progressed after B19 infection, and reported that immunocytes are infection target cells of B19 (Reference 10). It is known that expression of P antigen which is a receptor for B19 is low in these immunocytes, so that the possibility of B19 infection to immunocytes through a molecule other than P antigen protein was suggested.

### DISCLOSURE OF THE INVENTION

Problems which the Invention Tries to Solve

An object of the present invention is to provide a novel receptor for human parvovirus B19, other than P antigen, as well as a reagent for measuring human parvovirus B19, a reagent for adsorbing human parvovirus B19, and an agent for suppressing infection, which utilize the receptor for human parvovirus B19. Another object of the present invention is to provide means for suppressing infection by human parvovirus B19 by inhibiting the binding between the above-mentioned receptor for human parvovirus B19 and human parvovirus B19. Still another object of the present invention is to provide a process for producing a cell which presents the above-mentioned receptor for human parvovirus B19.

Means for Solving the Problem

The present inventors intensively studied to discover that Ku80 which is a protein having a molecular weight of 80 kDa, which was discovered as a target autoantigen corresponding to the autoantibody observed in systemic lupus erythematodes (SLE), is an infection receptor of human parvovirus B19, thereby completing the present invention.

That is, the present invention provides a receptor for human parvovirus B19, consisting essentially of a protein having the amino acid sequence shown in SEQ ID NO:1 in SEQUENCE LISTING, or a protein having the same amino acid sequence as shown in SEQ ID NO:1 except that a small number of amino acid residues are substituted or deleted, or a small number of amino acid residues are inserted or added, which protein binds to human parvovirus B19. The present invention also provides an agent for binding human parvovirus B19, consisting essentially of the above-mentioned receptor for human parvovirus B19 according to the present invention. The present invention further provides an agent for suppressing infection by human parvovirus B19, comprising as an effective ingredient a substance which inhibits binding between the receptor for human parvovirus B19 and human parvovirus B19, or a virus-binding fragment thereof. The present invention still further provides a process for producing a cell presenting the receptor for human parvovirus B19.

By the present invention, a novel B19 receptor was provided. Since the B19 receptor according to the present invention specifically binds to B19, it may be used as a reagent for measuring B19 or as an absorbent for B19. Further, since an antibody which undergoes antigen-antibody reaction with the B19 receptor inhibits the replication of B19, it may be used as an agent for suppressing infection by B19.

### BRIEF DESCRIPTION OF THE DRAWINGS

FIG. 1 shows the results of flow cytometry, showing the states of infection by B19 to various cell lines.

FIG. 2 shows the results of flow cytometry, showing the states of expression of P antigen in various cell lines.

FIG. 3 shows the results of flow cytometry, showing the binding of the recombinant B19 capsid (rB19ECP) to H9 cells.

FIG. 4 shows the mass spectrum of the protein binding to rB19ECP-Sepharose.

FIG. 5 shows the results of Western blot analysis of the rB19ECP-binding protein.

FIG. 6 shows the results of binding experiment between rB19ECP and biotinylated rKu80.

FIG. 7 shows the results of binding inhibition experiment (1) between rB19ECP and biotinylated rKu80.

FIG. 8 shows the results of binding inhibition experiment (2) between rB19ECP and biotinylated rKu80.

FIG. 9 shows the results of binding inhibition experiment (3) between rB19ECP and biotinylated rKu80.

FIG. 10 shows the results of binding inhibition experiment (4) between rB19ECP and biotinylated rKu80.

FIG. 11 shows the results of flow cytometry, showing the states of expression of Ku80 on the cell surfaces of various cell lines.

FIG. 12 shows the results of adsorption inhibition experiment of B19 to KU812Ep6 cell line.

FIG. 13 shows the results of replication inhibition experiment of B19 in KU812Ep6 cell line.

FIG. 14 shows the state of expression of Ku80 on the surfaces of bone marrow cells.

### BEST MODE FOR CARRYING OUT THE INVENTION

As described above, the present inventors discovered that Ku80 is a novel receptor for human parvovirus B19, other than P antigen (human parvovirus B19 will be hereinafter referred to as “B19” for short). Ku80 is a protein having a molecular weight of 80 kDa, which was discovered as the target autoantigen corresponding to the autoantibody observed in SLE cases. It is known that Ku80 is a protein localized in cells and functions as an intracellular protein. It is thought that Ku80 forms a heterodimer with Ku70 (Reference 19), and participates in repair and recombination of DNA as a regulatory factor of DNA-dependent protein kinase in cells (References 23 and 24). On the other hand, it has been reported that expression of Ku80 comes to be observed on the surfaces of vascular endothelial cells and RD cells which are human myosarcoma cell line under low oxygen environment, and that Ku80 is involved in adhesion of lymphocytic cells (References 25, 26 and 27). It has also been reported that Ku80 expressed on the surface of HGT-1 cell which is a human gastric carcinoma cell line functions as a somatostatin receptor and participates in signal transduction (Reference 28). As described in the above-mentioned reports, it is known that there are cases where Ku80 is expressed on cell surfaces and functions.

The nucleotide sequence of Ku80 gene and the amino acid sequence encoded thereby are shown in SEQ ID NO: 2 in SEQUENCE LISTING, and the amino acid sequence alone is shown in SEQ ID NO:1 (GenBank Accession No. M30938).

In general, it is well-known in the art that there are cases wherein the physiological activity of a physiologically active peptide is retained even if the amino acid sequence of the peptide is modified such that a small number of amino acids in the amino acid sequence are substituted, deleted, inserted or added. Therefore, a peptide having the same amino acid sequence as shown in SEQ ID NO:1 except that a small number of amino acids are substituted, deleted, inserted or added, which peptide has an ability to bind to B19 can also be used in the same manner as Ku80, so that it is also within the scope of the present invention. The term “a small number” is preferably one to several, or a peptide having an amino acid sequence having a homology of not less than 90%, more preferably not less than 95%, with the amino acid sequence shown in SEQ ID NO:1 is preferred. The homology of the amino acid sequence may easily be calculated by using a well-known software such as FASTA, and such a software is available on the internet. The 20 kinds of amino acids constituting naturally occurring proteins may be grouped into groups of similar properties, that is, for example, neutral amino acids having low polar side chains (Gly, Ile, Val, Leu, Ala, Met, Pro), neutral amino acids having hydrophilic side chains (Asn, Gln, Thr, Ser, Tyr, Cys), acidic amino acids (Asp, Glu), basic amino acids (Arg, Lys, His) and aromatic amino acids (Phe, Tyr, Trp). When substituting an amino acid with another amino acid, the property of the polypeptide is often not changed if the amino acid is substituted with an amino acid in the same group, so that it is preferred.

As experimentally confirmed in the Examples below, Ku80 functions as an infection receptor for B19, and B19 functions as a ligand of Ku80, so that Ku80 and B19 specifically bind. Therefore, Ku80 may be used as a specific agent for binding B19. The “agent for binding B19” is an agent for specifically binding B19 and is used for some use utilizing the specific binding with B19. Preferred examples of the uses include reagent for measuring B19, agent for adsorbing B19 and agent for suppressing infection by B19. Each of them will now be described in more detail.

Since the infection receptor for B19 according to the present invention specifically binds to B19, B19 may be measured using the receptor according to the present invention. The term “measure” includes both quantification and detection. This may be attained in the similar manner as immunoassays utilizing the specific binding between an antigen and an antibody (antigen-antibody reaction). For example, B19 in a sample may be measured by immobilizing the receptor according to the present invention on solid phase, contacting the sample containing B19 with the immobilized receptor, reacting the resultant with an anti-B19 antibody labeled with a fluorescent or enzyme marker after washing, and measuring the marker bound to the solid phase after washing. Since the receptor according to the present invention is a protein, the immobilization of the receptor on the solid phase may easily be attained by well-known methods, for example, by physical adsorption of the receptor to wells of a microtiter plate made of polystyrene or to a nitrocellulose filter, or by covalent bond through an amino group to a carrier having a functional group. As a method for detecting B19 directly using the infection receptor for B19, receptor-mediated hemagglutination assay (Reference 33) using P antigen as a ligand has been developed and is used for screening B19 in the blood to be transfused. By replacing P antigen with Ku80, a new receptor-mediated hemagglutination assay may be established. Since P antigen is a sugar chain antigen, it is difficult to chemically synthesize it. In contrast, Ku80 is a peptide and the nucleotide sequence of the gene encoding Ku80 is known, so that it may be produced as a recombinant protein in a large scale, and production of a fragment thereof is also easy.

Since the infection receptor for B19 according to the present invention specifically binds to B19, the receptor according to the present invention may also be used as an agent for adsorbing B19. Since B19 is a small virus, it is difficult to remove B19 with a filter. By passing a sample containing B19 through a filter to which the receptor according to the present invention is immobilized, or through a column containing a carrier packed therein, on which the receptor according to the present invention is immobilized, B19 may be removed. Further, since the B19 adsorbed to the immobilized receptor is liberated by a treatment such as treatment with urea or guanidine, or by change of pH or salt concentration, the above-mentioned immobilized receptor may be used for purification or concentration of B19.

Since the infection receptor for B19 according to the present invention specifically binds to B19, it can suppress the infection by B19 by inhibiting the binding between the receptor of the present invention and B19, so that it may be used as an agent for suppressing infection by B19. Further, by selecting a substance which inhibits the binding, using the receptor according to the present invention and B19, a substance which is used as an agent for suppressing infection by B19 may be discovered. Examples of the substance which inhibits the binding between the receptor according to the present invention and B19 include polypeptides derived from the receptor according to the present invention and virus-binding fragments thereof; antibodies which undergo antigen-antibody reaction with the receptor according to the present invention and antigen-binding fragments thereof; polypeptides derived from B19 and receptor-binding fragments thereof; and antibodies which undergo antigen-antibody reaction with B19 and antigen-binding fragments thereof, but the substance is not restricted thereto. The substance which inhibits the binding between the receptor according to the present invention and B19 may be, for example, selected by the following procedure: Purified Ku80, Ku80 produced by genetic engineering process or Ku80-expressing cells is immobilized on a solid phase. The substance to be selected is added thereto together with B19 or rB19ECP, and the mixture is allowed to react. After washing, the B19 or rB19ECP bound to the Ku80 on the solid phase is quantitatively measured by using an anti-B19 antibody, thereby discovering a substance which inhibits the binding between Ku80 and B19. Although the substance which inhibits the binding may be selected from various random libraries, the substances having high probabilities to inhibit the binding may be narrowed by the following procedure: For example, a column in which Ku80 is immobilized is prepared, and peptides obtained by fragmentation of B19 with a protease or the like are passed therethrough, and then the peptides are eluted after washing. The thus obtained peptides originated from B19, having binding abilities to Ku80 are the substances having high probabilities to inhibit the binding between Ku80 and B19. By immunizing an animal with these peptides originated from B19 by a conventional method, anti-B19 antibodies having high probabilities to inhibit the binding between Ku80 and B19 may be obtained.

The agent for suppressing infection according to the present invention means a pharmaceutical composition useful as a drug, comprising an antibody, preferably a humanized antibody or monoclonal antibody, preferably human monoclonal antibody, which binds to the receptor according to the present invention or to a fragment thereof, or a fragment of the antibody, and a pharmaceutically acceptable carrier. Examples of the pharmaceutically acceptable carrier include vehicles, diluents, extenders, disintegrating agents, stabilizers, preservatives, buffers, emulsifiers, aromatics, coloring agents, sweeteners, thickening agents, correctives, solubilizers and other additives. By using one or more of these carriers, pharmaceutical compositions in the forms of tablets, balls, powders, granules, injection solutions, liquids, capsules, elixirs, suspensions, emulsions and syrups may be formulated. These pharmaceutical compositions may be administered orally or parenterally. Other forms for parenteral administration include external liquids and suppositories for rectal administration, which contain one or more active ingredients and which are formulated by a conventional method. Although the dose of administration differs depending on the age, sex, body weight and symptoms of the patient, therapeutic effect, method for administration, treatment time and the active component (antibody or the like) contained in the infection suppressing agent as a pharmaceutical composition, usually, 10 μg to 1000 mg may be administered per time per an adult. However, since the dose of administration varies depending on various conditions, in some cases, a dose less than the above-mentioned dose may be sufficient, and in some cases, a dose more than the above-mentioned range may be necessary. An injection solution may be formulated by dissolving or suspending the active component in an atoxic and pharmaceutically acceptable liquid carrier such as physiological saline or commercially available distilled water for injections to a concentration of 0.1 μg/ml to 10 mg/ml.

The thus prepared injection solution may be administered to a human patient requiring the treatment at a dose of 1 μg to 100 mg, preferably 50 μg to 50 mg per 1 kg body weight per time, once to several times per day. Examples of the administration routes include those appropriate in medicine, such as intravenous injection, subcutaneous injection, intracutaneous injection, intramuscular injection and intraperitoneal injection. Intravenous injection is preferred. In some cases, the injection solution may be formulated with a non-aqueous diluent (e.g., propylene glycol, polyethylene glycol, a plant oil such as olive oil, or an alcohol such as ethanol), suspending agent or emulsifier. Sterilization of such an injection solution may be carried out by mechanical sterilization in which the solution is passed through a sterilization filter, addition of a sterilizer or by irradiation. The injection solution may be produced in the form to be formulated when use. That is, the solution may be made into a sterilized solid composition by freeze-drying technique or the like, and the composition may be used by being dissolved in sterile distilled water for injection or in other solvent before use. Since Ku80 contributes to the replication of B19, an antisense RNA or RNAi which inhibits the expression of Ku80 gene may be used for the suppression of infection by B19, and thus may be used for the therapy or prevention of the diseases mentioned above to which the above-described infection-suppressing agent is applied.

Since it was clarified that Ku80 is a receptor for B19 by the present invention, and since B19 well infects the cells which present both of Ku80 and P antigen and is well replicated therein, B19 may be proliferated efficiently by infecting B19 to B19-sensitive cells which presents both of Ku80 and P antigen. Production of B19 in a large scale is useful for the study of therapeutic drugs for B19 and preparation of anti-B19 antibodies.

B19-adsoptive or B19 infection-sensitive cell presenting B19 receptor according to the present invention and P antigen may be selected from lymphocytic cells, erythroblasts and cell lines available from cell banks or the like. Alternatively, the cell may be selected from the cells which are made to express the B19 receptor and P antigen by a genetic engineering method. The term “adsorptive” herein means that the cell specifically (i.e., by the receptor-ligand interaction) adsorbs B19. If adsorption is detected by an immunoassay such as ELISA as will be concretely described in Examples below, it is judged that the cell is adsorptive. The term “B19 infection-sensitive” herein means that B19 proliferates in the cell, that is, the copy number of the virus is increased in the cell. This may be confirmed by the quantitative PCR method as will be concretely described in Examples below. If the increase of the copy number is confirmed, it is judged that the cell is infection-sensitive.

The cell expressing B19 receptor and P antigen may be obtained by, for example, introducing Ku80 gene and P antigen-associated genes into the cell and expressing the genes. Since Ku80 gene is known to have the nucleotide sequence shown in SEQ ID NO:2, expression of Ku80 may be attained by introducing the Ku80 gene into a cell by a conventional genetic engineering method and expressing the gene in the cell. On the other hand, since P antigen is a sugar chain antigen, P antigen may be synthesized in the cell by introducing a series of glycosyltransferase genes necessary for the biosynthesis of P antigen, and expressing the genes. The cell expressing both Ku80 and P antigen may be obtained by introducing the Ku80 gene into the cell expressing P antigen in vivo or in cultured conditions; or by introducing P antigen-associated genes into a cell expressing Ku80; or by introducing the both genes into a cell which expresses none of Ku80 and P antigen. In view of simplicity because the number of genes to be introduced is small, it is preferred to introduce the Ku80 gene into a cell having P antigen.

The B19 infection-sensitive cell presenting the B19 receptor according to the present invention and P antigen may be distinguished and separated by, for example, fluorescent antibody technique, flow cytometry or the like using a commercially available anti-Ku80 antibody and an anti-P antigen antibody. Distinguishment and separation of the B19 infection-sensitive cells may be attained by distinguishing and separating cells using one of the anti-Ku80 antibody and the anti-P antigen antibody, and then subjecting the separated cells again to the same distinguishment/separation operation using the other antibody; or in one step by using a flow cytometer after treating the cells simultaneously with both of the antibodies.

Confirmation that the cell presenting the B19 receptor and P antigen, obtained by the above-described step, is sensitive to the infection by B19 may be attained as follows: After culturing the cell under ordinary culturing conditions, B19 is added to the culture medium. After culturing the cells for a prescribed period, the cells are harvested. Confirmation of the infection by B19 to the cells may be attained by qualititatively or quantitatively detect a B19 antigen produced in the cells due to the infection and proliferation of B19 by an immunoassay using an anti-B19 monoclonal antibody. Alternatively, confirmation of the infection may also be attained by detecting the B19 gene replicated in the cells by a molecular biological technique after infection by B19.

### EXAMPLES

The invention will now be described by way of Examples thereof. It should be noted that the present invention is not restricted to the Examples below.

1. Methods

1-1 Materials

(1) Cells

KU812Ep6 is a cell line of erythroblastic cell which is readily infected by B19, which was cloned from a chronic myelogenous leukemia cell line by the KU812 limiting dilution method in the presence of erythropoietin (Japanese Laid-open Patent Application (Kokai) No. 11-32757, Reference 16). Human T lymphocytic cell line H9 was furnished by ATCC, and human monocyte cell line U937, human colon adenocarcinoma cell line SW620 and human bladder cancer cell line T24 were firnished by Cell Resource Center for Biomedical Research, Institute of Development, Aging and Cancer, Tohoku University. KU812Ep6 was cultured in RPMI medium supplemented with 10% fetal bovine serum (FBS) and 6 IU/ml of erythropoietin (Kirin Brewery). H9, U937 and SW620 were cultured in RPMI medium supplemented with 10% FBS, and T24 was cultured in MEM medium supplemented with 10% FBS. The cultures were carried out at 37° C. under 5% CO₂.

Bone marrow blood monocytes were collected from the samples from patients who received clinical tests for fervescence, anemia or the like (excluding hematopoietic tumors), with consent of the patients. Bone marrow blood monocytes were separated from the obtained bone marrow samples by specific gravity centrifugation method using Ficoll-Hypaque (Pharmacia), and cultured in RPMI medium supplemented with 1 IU/ml of erythropoietin (Kirin Brewery) and 10% FBS.

(2) Human Parvovirus B19

The serum collected from a patient suffering from acute B19 infectious disease was used as the B19 virus. The serum contained 2×10¹⁴ copies of B19 virus, but anti-B19 M antibody and anti-B19 IgG antibody were not detected. As the control, the serum from a healthy individual who has never been infected by B19, in which B19 DNA, anti-B19 IgM antibody and anti-B19 IgG antibody were not detected. The sera were stored at −80° C. until immediately before use.

In the binding inhibition experiments, intact B19 virus purified by column chromatography from a B19-positive serum, which was confirmed to be infective, was used (Reference 17).

(3) Antibodies

Antibody PAR3 (mouse, monoclonal) which recognizes VP1 that is a structural protein of B19 was a gift from Dr. Sugamura, Department of Immunology, Tohoku University. Anti-Ku80 antibody (mouse, monoclonal) which recognizes the N-terminal of Ku80 was obtained from Oncogene, and anti-Ku80 antibody (mouse, monoclonal) which recognizes the C-terminal was obtained from Pharmingen. Anti-Ku70 antibody (mouse, monoclonal) and anti-CD106 antibody (mouse, monoclonal) were obtained from Pharmingen, and GL4 which is an anti-globoside (P antigen) antibody (rabbit, polyclonal) was obtained from Matreya. 1F5 is an anti-idiotypic monoclonal antibody (Reference 18) against 08-1 idiotype of human anti-DNA antibody, and was used as a negative control. PE-labeled anti-rabbit antibody, PE-labeled anti-Glycophorin A antibody, PE-labeled anti-CD3 antibody, PE-labeled anti-CD20 antibody, PE-labeled anti-CD14 antibody and PE-labeled anti-CD56 antibody were obtained from Nippon Becton Dickinson.

(4) Recombinant Proteins

Recombinant Ku80 (rKu80) and recombinant Ku70 (rKu70) were gifts from Dr. Mitsumori (Kyoto University) (Reference 19). Soluble CD26 (sCD26) was a gift from Dr. Morimoto (University of Tokyo) (Reference 20). Recombinant B19 empty capsid protein (rB19ECP) was a gift from Denka Seiken (References 21 and 22).

Biotinylation of rB19ECP was performed by reacting rB19ECP with sulfo-LC-biotin (Pierce) on ice for 2 hours. The non-reacted sulfo-LC-biotin was removed by dialysis against PBS. By the same method, bovine serum albumin (BSA) was biotinylated and used as a control.

rB19ECP-bound Sepharose (rB19ECP-Sepharose) was prepared using CNBr-activated Sepharose (Pharmacia Biotech) and rB19ECP in accordance with the protocol. As a control, BSA-Sepharose was prepared.

1-2. Infection by B19 In Vitro

To each of the suspensions of various infection target cells (1×10⁵ cells/100 μl culture medium), 2×10¹⁰ copies of B19 virus were added and the cells were allowed to adsorb the virus for 1 hour on ice. After removing the excessive B19 by washing 4 times, DNAs were extracted and the number of copies of B19 was measured by quantitative PCR. In the infection experiments, DNAs were extracted after culturing the cells at 37° C. under 5% CO₂ for 2 days, and B19 virus was quantified. In the inhibition experiments, each of the various antibodies were allowed to react for 1 hour on ice before the adsorption of B19.

1-3. Measurement of B19 DNA

DNA extraction solution was added to the cells and the culture medium to attain final concentrations of 10 mM Tris (pH7.6), 1 mM EDTA, 50 mM NaCl and 0.5% SDS, and the resulting mixture was treated with protease K (0.2 μg/ml) at 37° C. for 24 hours, followed by DNA extraction by the phenol-chloroform method. The extracted DNAs were dissolved in 10 mM Tris (pH7.6) supplemented with 0.1 mM EDTA.

The B19 virus was quantified, using “TaqMan PCR Reagent Kit”, by quantitative PCR amplifying the VP1 region (nt.2598-2752) which is a structural protein gene of B19 virus genome. To a DNA sample (0.5 μg) which has not been measured, were added dUTP (400 μM), dATP (200 μM), dCTP (200 μM), dGTP (200 μM), MgCl₂ (3.5 mM), forward primer (200 nM), reverse primer (200 nM), probe (100 nM), Amp Erase UNG (0.01 U/μl) and Ampli Taq Gold (0.025 U/μl), and then TaqMan buffer was added to a total volume of 50 μl, followed by allowing the mixture to react. The nucleotide sequence of the forward primer was 5′-ccctagaaaacccatcctctgtg-3′ and the nucleotide sequence of the reverse primer was 5′-aggttctgcatgactgctactgg-3′. As the probe for detection, a DNA fragment labeled with a fluorescent dye FAM, having the nucleotide sequence of 5′-tcatggacagttatctgaccaccccca-3′, which recognizes nt. 2692-2718 of VP1 gene in B19 genome, was used. The amplification was carried out by repeating 40 times a cycle of 95° C. for 15 seconds and 60° C. for 1 minute, after reaction at 50° C. for 2 minutes and at 95° C. for 10 minutes. All reactions were carried out using ABI/PRISM 7700 Sequence Detector System.

1-4. Identification of B19-Binding Molecule

(1) Binding of B19 to Cells

To the cell suspension in PBS, biotinylated rB19ECP was added, and the resulting mixture was allowed to react for 30 minutes on ice. After washing the cells with PBS, avidin-FITC (Sigma) was added and the resulting mixture was allowed to react in the same manner, followed by analyzing the reaction product with FACS Caliber (Becton Dickinson).

(2) Identification of B19-Binding Protein

To about 1×10⁶H9 cells, sulfo-LC-biotin (Pierce) was added and the resulting mixture was allowed to react at room temperature for 30 minutes to biotinylate the surface of the H9 cells. The non-reacted biotin was removed by washing the cells with cold PBS three times, and the cells were suspended in a cell lysis solution (100 mM NaCl, 1% TritonX-100, 1 mM MgCl₂, 20 mM Tris (pH7.6), 2 mM PMSF). After reaction at 4° C. for 90 minutes, the components extracted from nuclei were removed by centrifugation to obtain a cell lysate. The cell lysate and Sepharose were reacted at 4° C. for 24 hours under gentle stirring, and the proteins non-specifically bound to the Sepharose was removed. After centrifugation, rB19ECP-Sepharose was added to the supernatant, and the resultant was allowed to react at 4° C. for 2 hours under gentle stirring. After washing rB19ECP-Sepharose 3 times with cold washing solution (20 mM Tris (pH7.6), 0.1% Triton X-100, 1 mM MgCl₂, 1 mM CaCl₂), sample buffer (0.125M Tris-HCl, 10% 2-mercaptoethanol, 4% SDS, 10% sucrose, 0.004% Bromphenol Blue) was added thereto, and the resulting mixture was boiled for 5 minutes.

The protein bound to rB19ECP was separated by 7.5% SDS-polyacrylamide gel electrophoresis and was transferred to a PVDF membrane. After blocking the membrane with 1% skim milk at room temperature for 1 hour, the membrane was reacted with avidin-HRP at room temperature for 1 hour, and then the biotinylated protein was detected using ECL kit (Pharmacia).

For identification of the protein, without biotinylating the cell surfaces, the cell lysate and rB19ECP-Sepharose were reacted in the same manner, and the protein binding to rB19ECP was separated. After electrophoresis on 7.5% gel, the protein was stained with CBB dyeing solution (0.1% SDS, 0.25% Coomassie brilliant blue R250, 45% ethanol, 10% acetic acid solution), and the gel containing the desired protein was cut off. The protein was digested with a lysyl endopeptidase, and the resultant was subjected to MALDI-TOF MS analysis.

(3) Western Blotting Analysis

The rB19ECP-binding protein separated by the above-described method or the protein immunoprecipitated with anti-Ku80 antibody was transferred to a PVDF membrane, and the membrane was blocked with 1% skim milk at room temperature for 1 hour. Anti-Ku80 antibody was diluted with PBS containing 0.1% Tween 20 to a final concentration of 0.5 μg/ml, and the resultant was reacted with the PVDF membrane at room temperature for 1 hour under shaking. Thereafter, the membrane was washed with PBS containing 0.1% Tween 20, and then the membrane was reacted with peroxidase-labeled anti-mouse antibody (1:2000) which was a secondary antibody at room temperature for 1 hour. Detection of the chemiluminescence was carried out using ECL detection reagent (Amarsham Pharmacia Biotech).

1-5. Binding-Inhibition Experiment by ELISA

Enzyme-linked immunosorbent assay (ELISA) was carried out using a plate on which rB19ECP was immobilized, included in Parvo IgG-EIA Seiken kit (Denka Seiken). The optimum biotinylated recombinant Ku80 was reacted with the plate in the presence of one of various competing substances at room temperature for 45 minutes, and the plate was then washed with the washing solution included in the kit. The plate was then allowed to react with avidin-HRP (1:1000) at room temperature for 45 minutes, and the bound label was detected using the substrate. As a negative control, biotinylated BSA was used.

1-6. Flow Cytometry Analysis

To examine the state of infection by B19 in each cell line, flow cytometry analysis was performed. To the culture medium of the target cells for infection, B19-positive serum was added at a rate of 1:1000, and the resultant was cultured for 48 hours. After washing the infected cells with PBS, the cells were fixed with 4% paraformaldehyde, and then subjected to cell membrane-permeation treatment with Hanks' solution containing 0.1% saponin and 0.05% NaN₃. Then PAR3, an anti-B19-VP1 antibody, was added, and the resultant was allowed to react on ice for 30 minutes. The resultant was washed with PBS and then reacted with FITC-labeled anti-mouse IgG antibody (Sigma) in the similar manner.

Detection of the antigens on the cell surfaces were carried out by adding a primary antibody (5 μg/ml) to the cells suspended in PBS, allowing reaction on ice for 30 minutes, washing the cells with PBS, and then reacting FITC-labeled anti-mouse IgG antibody or PE-labeled anti-rabbit antibody (Jackson Immuno Research) on ice for 30 minutes.

Bone marrow cells were double-stained by staining the cells with anti-Ku80 antibody and FITC-labeled anti-mouse antibody, and reacting PE-labeled monoclonal antibody therewith on ice for 30 minutes.

All analyses were carried out with FACS Caliber (Becton Dickinson).

1-7. Staining with Fluorescent Antibody

(1) Detection of Infection by B19

Infected cells cultured for 48 hours in the presence of B19-positive serum (1:1000) for 48 hours were washed with PBS and mounted on a slide glass, followed by drying the cells in the air. The cells were then fixed with acetone-methanol (1:1) at −20° C. for 20 minutes, and then reacted with PAR3, an anti-B19-VP1 antibody, at 37° C. for 30 minutes. The cells were then washed with PBS and then reacted with biotin-labeled anti-mouse IgG antibody (SIGMA) (1:500) in the similar manner. Then avidin-FITC (1:200) was reacted at 37° C. for 30 minutes, and the resultant was observed with a fluorescence microscope.

(2) Binding Between B19ECP and KU812Ep6

Biotinylated rB19rECP was added to Ku812Ep6 cells, and the resultant was allowed to react for 1 hour on ice. After washing the cells with PBS, anti-Ku80 antibody (5 μg/ml) was added, and the mixture was allowed to react at room temperature for 30 minutes. To detect biotinylated rB19ECP, avidin-FITC (1:100) was added, and to detect anti-Ku80 antibody, TRITC-labeled anti-mouse IgG antibody (1:50) was added, followed by allowing the resultant to react at room temperature for 30 minutes. After the dyeing, the cells were mounted on a slide glass and observed with a fluorescence microscope.

2. Results

2-1. Adsorption and Infection Experiments of B19 to Cell Lines

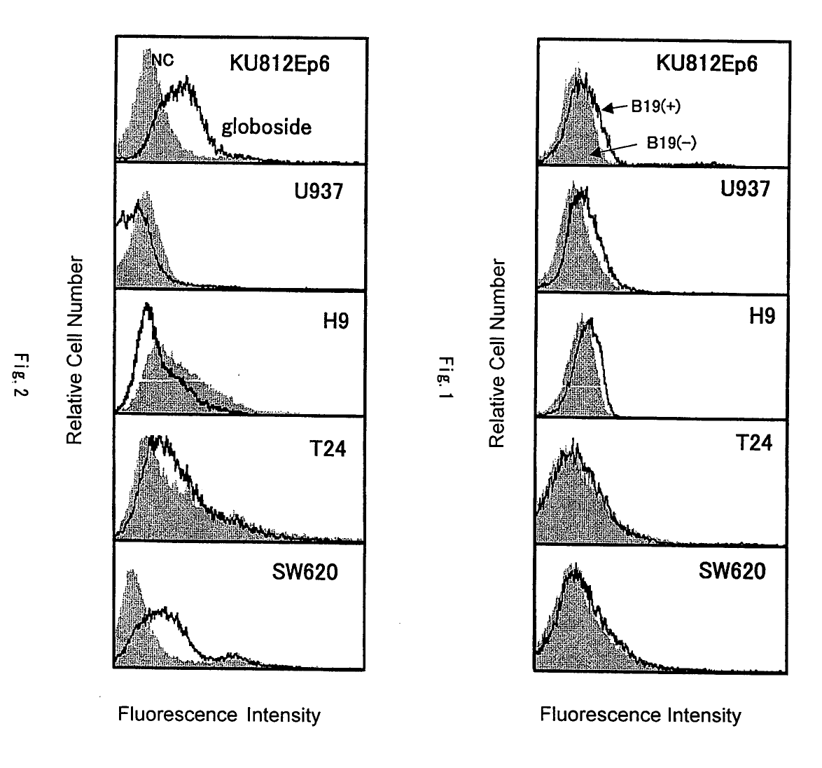

To investigate the state of infection in vitro by B19 to various cell lines including immunocytes, B19-positive serum was added to each of the culture media of the cell lines, and the cells were cultured for 48 hours, followed by detection of B19-VP1 protein on the cell surfaces and in the cells by flow cytometry. With the Ku812Ep6 cell lines known to exhibit high sensitivity to B19 infection, the cells were separated into two types of cell populations, that is, a cell population highly positive to anti-VP1 antibody, and a cell population weakly positive to anti-VP1 antibody (FIG. 1). The highly positive cell population was observed only in KU812Ep6, and weakly positive cell population alone was observed in macrophage cell line U937 and T lymphocytic cell line H9. On the other hand, neither weakly positive cells nor highly positive cells were observed in human bladder cancer cell line T24 and human colon adenocarcinoma cell line SW620. By detecting the B19 structural protein VP1 by fluorescent antibody technique, highly positive cells were observed in Ku812Ep6 alone (FIG. 1).

Using the cell lines, adsorption and infection of B19 were studied in relation to expression of P antigen. To each of the cell lines Ku812Ep6, U937 and H9, 13, 9 and 8 copies of B19, respectively, were adsorbed per cell. However, to T24 and SW620, 0.2 copies of B19 DNA were detected. Thus, prominent difference in adsorption of B19 was observed among the cell lines. On the other hand, in infection experiments, prominent replication of B19 was observed in KU812Ep6 alone, and significant increase in the copy number was not observed in other cell lines (Table 1).

TABLE 1

| | | Expression of P | B19 | |
|---|---|---|---|---|
| | | antigen on Cell | Adsorp- | |
| Cell Lines | Origin | Surfaces | tion | Replication |
| KU812Ep6 | Erythroblast | ++ | ++ | +++ |
| U937 | Monocyte | − | + | ± to − |
| H9 | T cell | − | + | ± to − |
| T24 | Bladder | + | ± | ± |
| | Epithelial | | | |
| | Cancer | | | |
| SW620 | Colon | + | ± | ± |
| | Adenocarcinoma | | | |

Expression of P antigen in these cell lines was examined by flow cytometry.

As a result, expression of P antigen was observed on the cell surfaces of KU812Ep6, T24 and SW620. However, expression of P antigen was not observed on H9 and U937 (FIG. 2). Since expression of P antigen and replication of B19 were not coincident because replication of B19 was observed in KU812Ep6 alone among KU812Ep6, T24 and SW620 in which expression of P antigen was observed, and since adsorption of B19 was observed on H9 and U937 cells on which P antigen is not expressed, existence of a molecule other than P antigen, which is involved in adsorption of B19 and infection by B19 was suggested.
2-2. Binding of Recombinant B19 Empty Capsid Protein (rB19ECP) to H₉Cell Surface

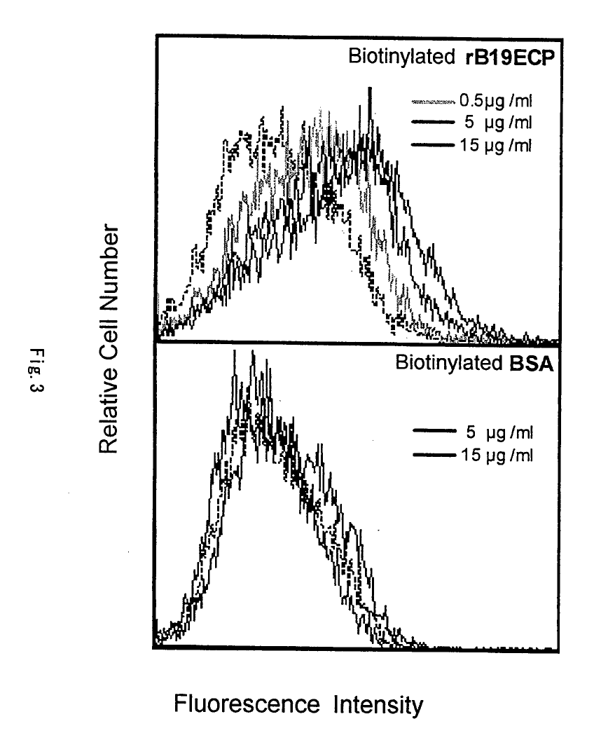

Using the T lymphocytic cell line H9 which exhibits B19 adsorption comparable to that by KU812Ep6 in spite of the fact that expression of P antigen is not observed, it was tried to identify the rB19ECP-binding protein. Biotinylated rB19ECP was bound to the cell surfaces of H9 concentration-dependently. On the other hand, binding of biotinylated BSA, which was a control, to the cell surfaces of H9 was not observed (FIG. 3).

2-3. Separation and Identification of rB19ECP-Binding Protein

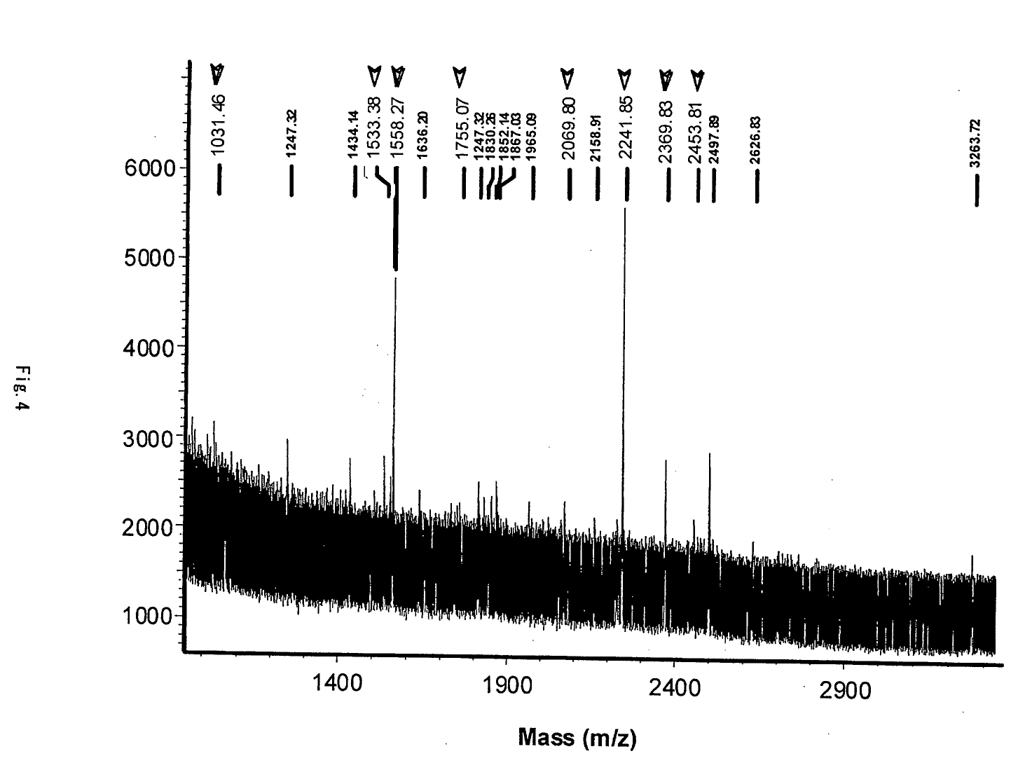

The surfaces of H9 cells were biotinylated, and a cell lysate thereof was prepared by the method described in 4.4(2). The cell lysate was reacted with rB19EPC-Sepharose, and separation and identification of the protein which bound to rB19ECP were tried. The protein which was bound to rB19ECP-Sepharose and precipitated was observed in the vicinity of 80 kDa in SDS polyacrylamide gel electrophoresis. On the other hand, with BSA-Sepharose used as a control, the protein was not observed. The protein of 80 kDa was digested within the gel with a lysyl endopeptidase, and the digestion product was analyzed by MALDI-TOF MS technique (FIG. 4). Homology search was performed on two databases SwissProt and NCBInr. As a result, it was thought that the protein of 80 kDa was Ku80 with high probability.

2-4. Confirmation of rB19ECP-Binding Protein by Western Blotting Analysis

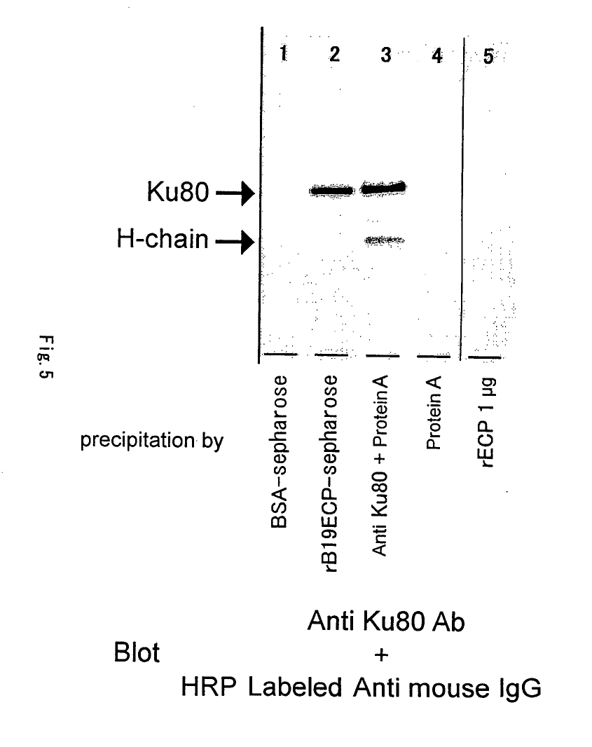

The protein of 80 kDa precipitated with rB19ECP-Sepharose reacted with anti-Ku80 antibody in Western blotting analysis. This protein was identical with the protein immunoprecipitated from H9 cell lysate with anti-Ku80 antibody. Thus, it was proved that the protein of 80 kDa bound to and precipitated with rB19ECP-Sepharose was Ku80 antigen (FIG. 5).

2-5. Confirmation Experiments about Binding Between B19 and Ku Antigen

1) Binding-Inhibition Experiments by Recombinant Protein

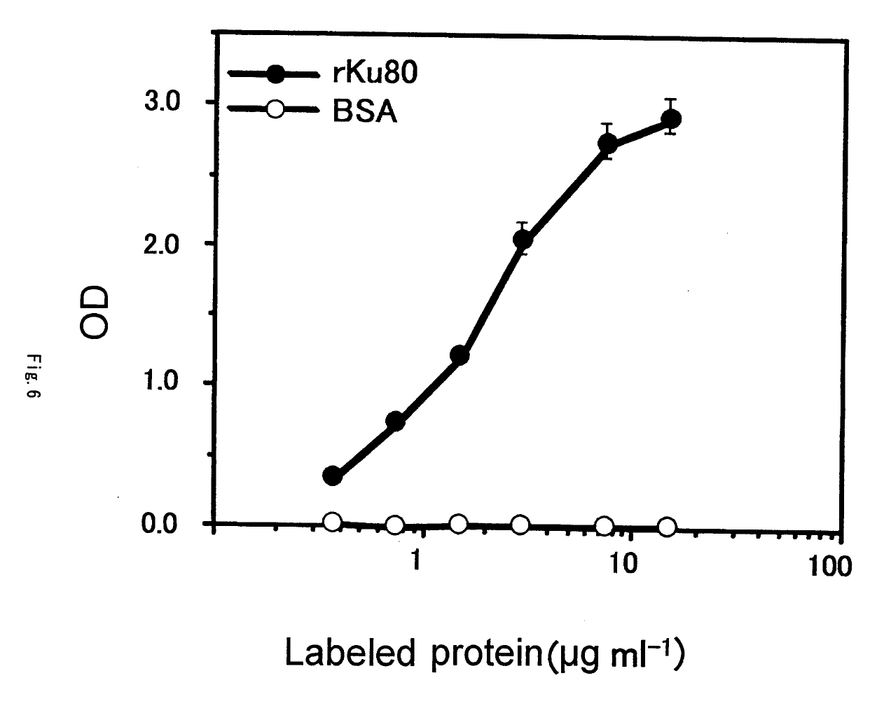

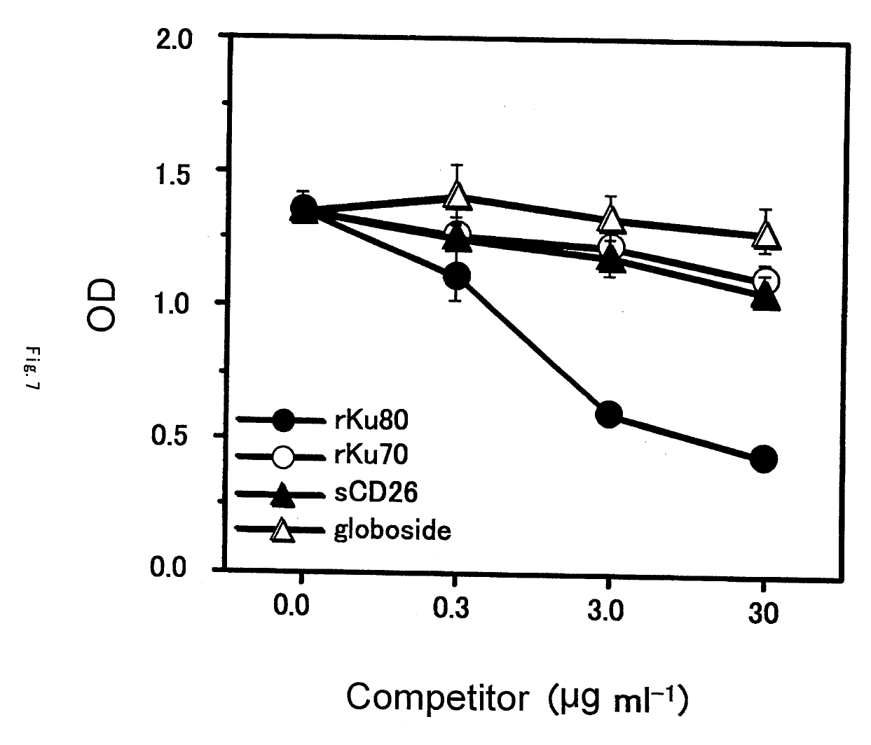

Biotinylated rKu80 bound to immobilized rB19ECP concentration-dependently (FIG. 6). This binding was specifically inhibited by non-labeled rKu80, and inhibition by rKu70 or solubilized CD26 used as a control was not observed (FIG. 207).

2) Binding-Inhibition Experiments by Purified B19 Virus

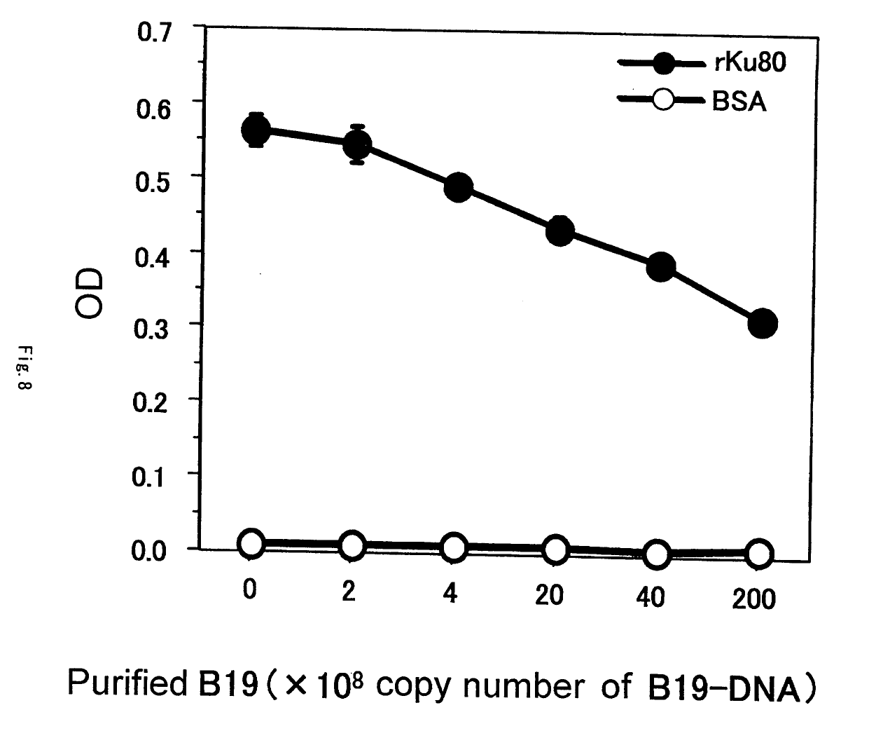

Binding of biotinylated rKu80 to immobilized rB19ECP was also inhibited by the B19 virus purified from a serum from a patient suffering from acute infectious disease of B19 concentration-dependently (FIG. 8).

3) Binding-Inhibition Experiments by Various Antibodies

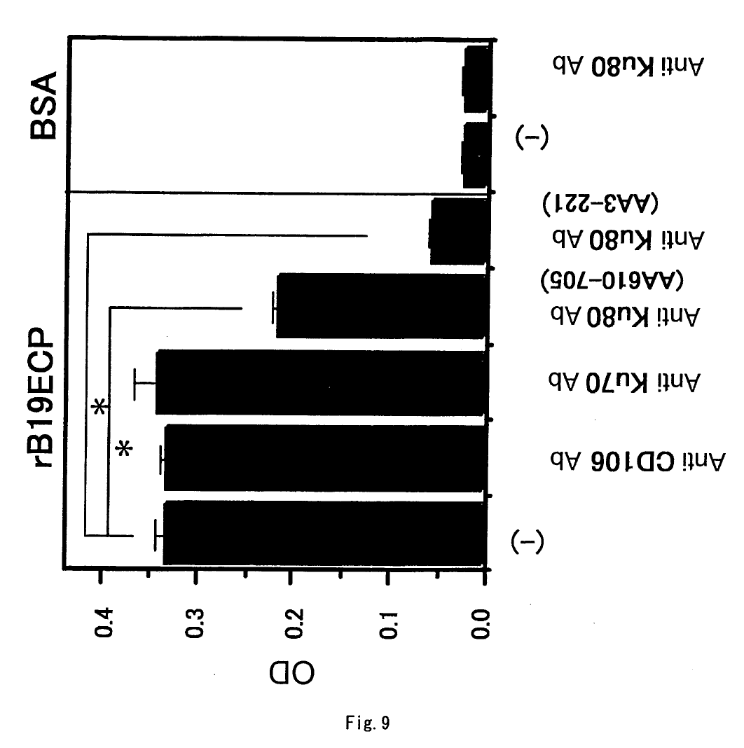

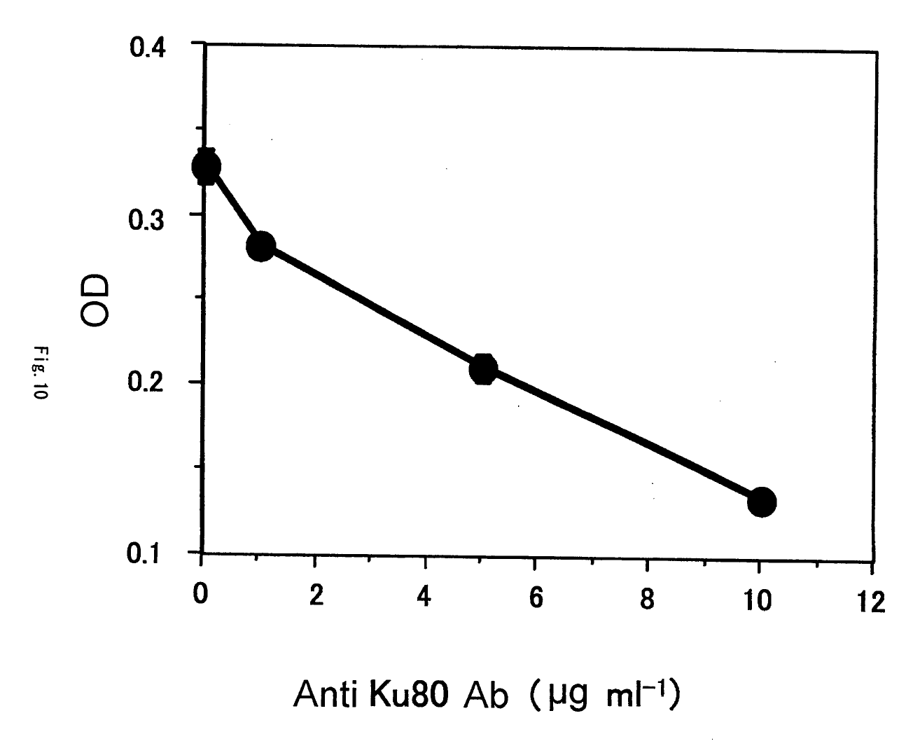

Binding of biotinylated rKu80 to immobilized rB19ECP was inhibited by anti-Ku80 antibody, but not by anti-Ku70 antibody or anti-CD106 antibody (FIG. 9). The inhibition by anti-Ku80 antibody was concentration-dependent (FIG. 10).

2-6. Expression of Ku80 Antigen on Surfaces of Various Cell Lines

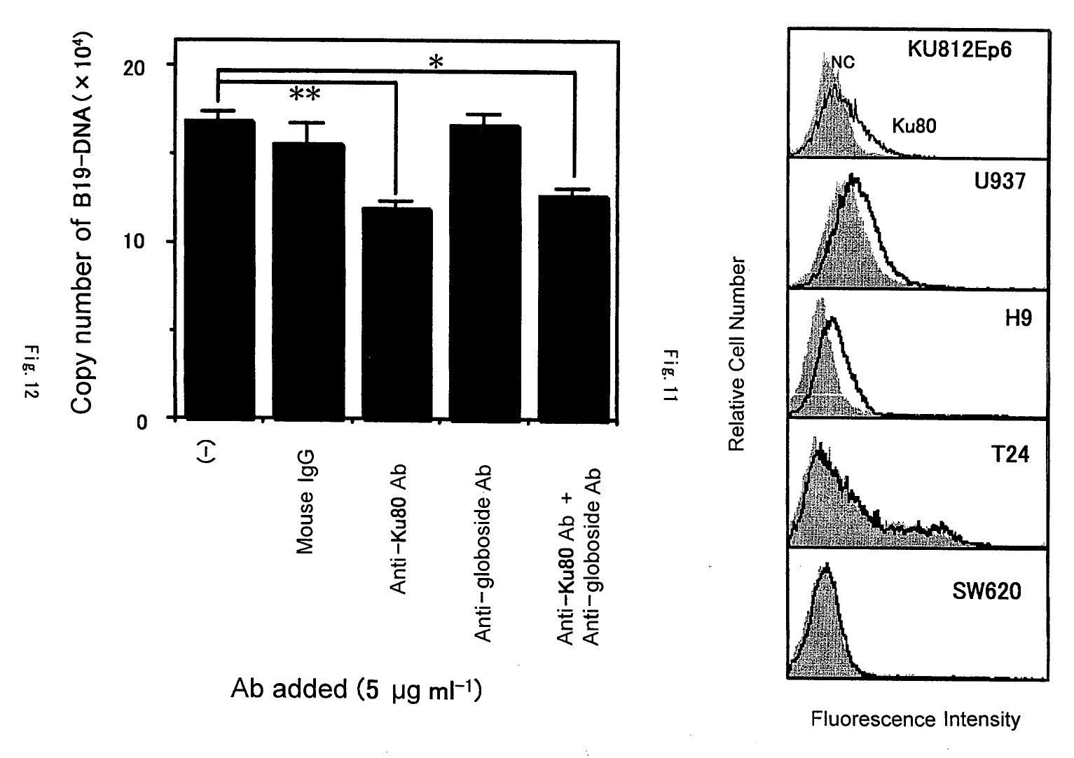

Expression of Ku80 antigen on the cell surfaces of KU812Ep6, H9 and U937 cell lines was observed by flow cytometry. On the other hand, expression of Ku80 antigen on the cell surfaces of T24 and SW620 cell lines was not observed (FIG. 11, Table 2).

TABLE 2

| | | Expression | | | Expression of |
|---|---|---|---|---|---|
| | | of P antigen | | | Ku80 antigen |
| | | on Cell | B19 | | on Cell |
| Cell Lines | Origin | Surfaces | Adsorption | Replication | Surfaces |
| KU812Ep6 | Erythroblast | ++ | ++ | +++ | + |
| U937 | Monocyte | − | + | ± to − | + |
| H9 | T cell | − | + | ± to − | + |
| T24 | Bladder | + | ± | ± | − |
| | Epithelial | | | | |
| | Cancer | | | | |
| SW620 | Colon Adeno- | + | ± | ± | − |
| | carcinoma | | | | |

2-7. Binding of B19 to KU812Ep6 Cell and Expression of Ku80

Using fluorescent antibody staining, binding of rB19ECP to KU812Ep6 cell and expression of Ku80 on the cell surface were examined. Cells on which localizations of rB19ECP and Ku80 were coincident were observed with a confocal microscope (FIG. 4B).

2-8. P Antigen and Ku Antigen in B19 Infection

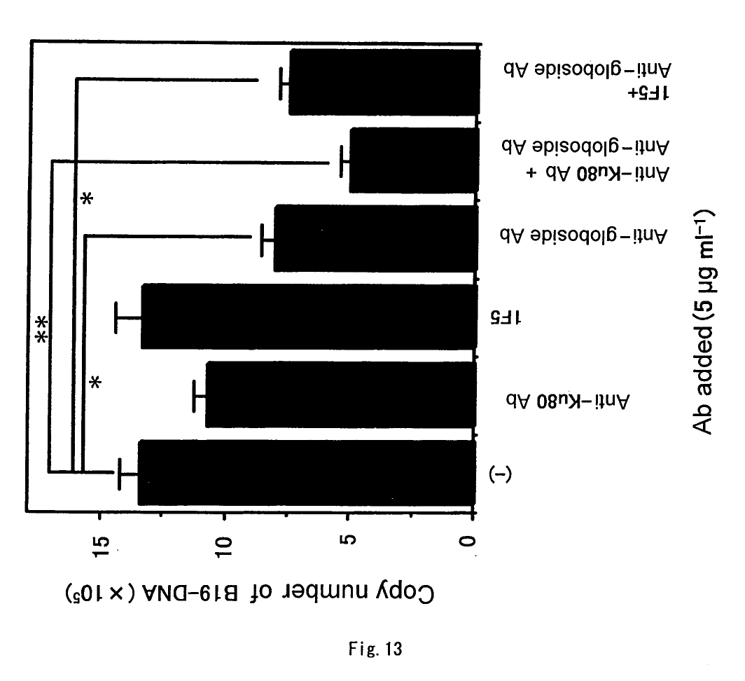

To examine the role of the identified Ku antigen in B19 infection, using KU812Ep6 which expresses P antigen and Ku antigen, and has an ability to replicate B19, the actions of a specific antibody in the adsorption and infection experiments of B19 were studied. Significant inhibition of the adsorption of B19 to Ku812Ep6 by anti-Ku80 antibody was observed, but the inhibition by anti-globoside antibody was not clear (FIG. 12). On the other hand, after culturing the cells for 2 days, replication of B19 in the cells was checked by quantitative PCR. As a result, the replication of B19 was inhibited in the presence of either anti-Ku80 antibody or anti-globoside (P antigen) antibody. In the presence of both of the antibodies, the inhibition ratio of the replication of B19 was increased (FIG. 13).

2-9. Expression of Ku80 Antigen in Bone Marrow Blood

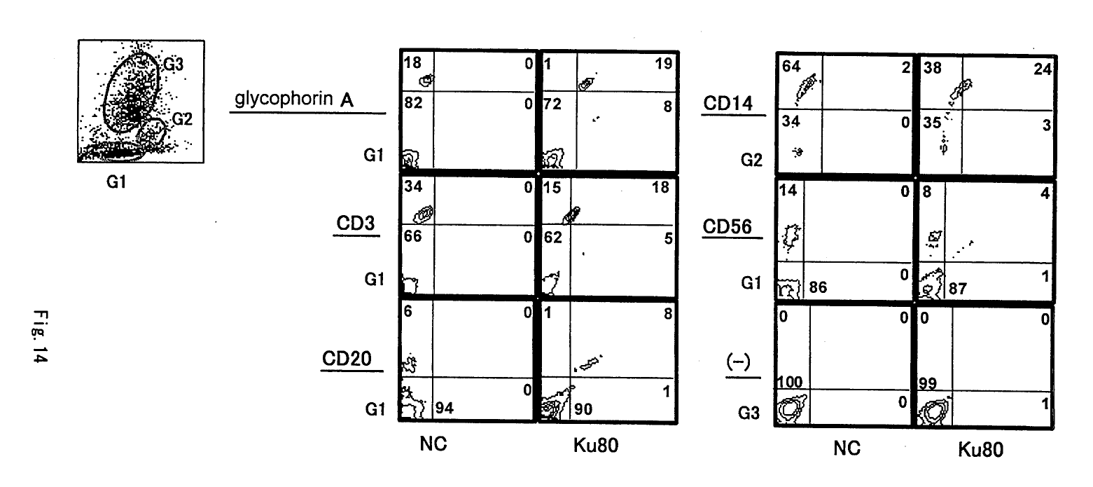

To determine whether Ku80 antibody is expressed in vivo on the bone marrow cells including B19-replicating cells or not, the expression of Ku80 on the surfaces of the cells in the bone marrow blood was analyzed by flow cytometry. As a result, strong expression of Ku80 was observed on the erythroblastic cells expressing Glycophorin A. Further, expression of Ku80 was observed on the cells which were CD20-, CD3- or CD14-positive, that are the cell surface markers of B cells, T cells and monocytes, respectively (FIG. 14).

2-10. B19 Infection to Bone Marrow Cells and P and Ku Antigens

Using a bone marrow sample, the manner of involvement of Ku80 and P antigens in B19 infection was studied in the same manner as for the cell lines. In the presence of anti-Ku80 antibody or anti-globoside antibody, 99.0% or 99.9% replication of B19 virus was inhibited, respectively. However, significant synergistic effect on the inhibition rate of replication was not observed even when both of the anti-Ku80 antibody and the anti-globoside antibody were added, in comparison with the cases where anti-Ku80 antibody or anti-globoside antibody was used individually (Table 3).

TABLE 3

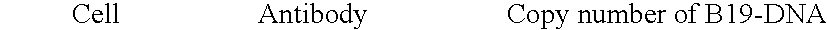

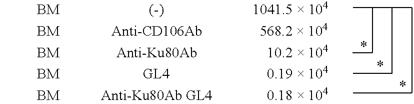

*P < 0.01

3. Discussion

By the present study, Ku80 was identified as a B19 infection-related molecule which is different from P antigen, in relation to the binding of B19 to cell surface. Ku80 was identified by MALDI-TOF MS analysis of the precipitated molecule which binds to recombinant B19 empty capsid protein (rB19ECP) from a T lymphocytic cell line H9 with which adsorption of B19 is observed but expression of P antigen is not observed. Adsorption of B19 to H9 cell is inhibited by 60% in the presence of 5 μg/ml of anti-Ku80 antibody (data not shown), and rB19ECP-binding molecule reacted with anti-Ku80 antibody in Western blot using anti-Ku80 antibody. Further, by the results of competitive ELISA using rB19ECP and rKu80, it was shown that the molecule which bound to rB19ECP was Ku80 and the binding between B19 and Ku80 is specific.

Ku80 is a protein having a molecular weight of 80 kDa, which was found as an target autoantigen of the autoantibody recognized in SLE cases. Ku80 is known to localize and to function as an intracellular protein. Ku80 forms a heterodimer with Ku70 (Reference 19), and is thought to be involved in DNA repair and recombination in cells as a regulatory factor of DNA-dependent protein kinase (References 23 and 24). On the other hand, it has been reported that expression of Ku80 comes to be observed on the surfaces of vascular endothelial cells and RD cells which are human myosarcoma cell line under low oxygen environment, and that Ku80 is involved in adhesion of lymphocytic cells (References 25, 26 and 27). It has also been reported that Ku80 expressed on the surface of HGT-1 cell which is a human gastric carcinoma cell line functions as a somatostatin receptor and participates in signal transduction (Reference 28). As described in the above-mentioned reports, it is known that there are cases where Ku80 is expressed on cell surfaces and functions.

In the present study, expression of Ku80 on the cell surface was observed in any of KU812Ep6, H9 and U937 which were observed to adsorb B19. Since P antigen which is a B19 receptor was not detected in H9 and U937, it is assumed that Ku80 plays an important role for the adsorption of B19 to these cell lines. Further, expression of Ku80 was examined on blood cells from the body, and a cell population of peripheral blood monocytes on which the expression of Ku80 on the cell surface detectable by anti-Ku80 antibody was not observed (data not shown). However, with the cells originated from bone marrow, expression of Ku80 on the surfaces of Glycophorin A-positive cells, CD14-positive cells, CD3-positive cells and CD20-positive cells, which are markers of erythroblasts, monocytes, T cells and B cells, respectively, was observed. Expression of Ku80 on the surfaces of these cells may be attributed to the fact that the physiological environment of the bone marrow cells is a low-oxygen environment. It is thought that the oxygen partial pressure in the bone marrow is 5 to 7% O₂ (37-52 mmHg) (References 29 and 30). Recently, it was reported that replication of the infected B19 is promoted in low oxygen environment (Reference 31). Further, although the mechanism of promotion of replication of the infected B19 under low oxygen is not known at present, there is a possibility that the infection efficiency of B19 is increased and so the replication of B19 is promoted because Ku80 is presented on the cell surfaces, which is intrinsically localized inside the cells.

The conditions and the mechanism by which Ku80 which is an intracellular protein is presented on the cell surface have not been well understood. To understand the mechanism of expression of Ku80 on the cell surface is important to the understanding of the infection by B19, and further studies are required.

The role of Ku80 in the infection by B19 was studied by the B19 adsorption and infection-inhibition experiments using Ku812Ep6 which is a cell line sensitive to B19 infection. Inhibition of adsorption of B19 was observed only in the presence of the anti-Ku80 antibody. The B19 replication-inhibiting effect in the presence of anti-Ku80 antibody and anti-globoside antibody were about 20% and 40%, respectively. Although the difference in the action of anti-globoside antibody was observed, this may be because the amount of the anti-globoside antibody was not sufficient for inhibiting the binding between B19 and globoside. However, in the competitive ELISA, the binding between B19 and Ku80 was not inhibited by globoside, so that it is thought that the binding sites in B19 for the binding to Ku80 and globoside are different. From this, it is thought that there is a possibility that the copy number of adsorbed B19 did not apparently change because the B19 which could not bind to globoside by the anti-globoside antibody bound to Ku80. On the other hand, in the infection experiments. inhibition of B19 replication by anti-globoside antibody was observed. This presumably indicates that the infection efficiency of B19 through globoside is higher than the infection efficiency through Ku80. In the presence of both of anti-Ku80 antibody and anti-globoside antibody, the B19 replication-inhibiting effect was increased up to about 60%, so that anti-Ku80 antibody exhibited synergistic inhibition effect to anti-globoside antibody.

In the B19 infection experiments using bone marrow cells, the inhibition of B19 replication by anti-Ku80 antibody alone was 99.0%. This inhibition rate was about the same as that when the anti-globoside antibody was added. Although the synergistic effect for the inhibition of B19 replication in the presence of both of the anti-globoside antibody and anti-Ku80 antibody was not observed, this may be because of the difference in expressed amounts of globoside and Ku80 antigens, and of the difference in infection efficienencs of B19 by globoside and Ku80. Bone marrow erythroblastic cells highly express globoside. Further, as indicated by the results shown in FIG. 4C and FIG. 4D, it is suggested that the infection efficiency of B19 through globoside is higher than that through Ku80. Therefore, the results of the infection experiments using bone marrow cells reflect the infection by B19 to erythroblasts maily through globoside. Therefore, it is thought that in the presence of anti-globoside antibody, the main infection by B19 to erythroblasts was efficiently inhibited, and was difficult to observe the participation of Ku80 to the infection by B19.

From the results shown in Table 1, it is thought that Ku80 plays an important role in the adsorption of B19 because B19 is adsorbed on the cells on which Ku80 alone is positive, because the amount of adsorbed B19 is small when the expression of Ku80 was negative even to the globoside-positive cells, and because the inhibition of adsorption of B19 is observed by anti-Ku80 antibody alone in the experiments using cell lines. Further, since it was observed that inhibition of B19 replication was observed by anti-Ku80 antibody alone in the infection experiments using the cell lines and bone marrow cells, and since anti-Ku80 antibody can exhibit synergistic inhibition effect with anti-globoside antibody, there is a possibility that expression of Ku80 on cell surfaces may influence on B19 infection and define B19 infection. That is, it is assumed that Ku80 functions as a B19 infection-related molecule and plays a role as a B19 infection receptor or a co-receptor promoting the infection efficiency.

By the present invention, it was shown that Ku80 is expressed in vivo on the surfaces of T cells, B cells and monocytes, in addition to the erythroblastic cells in bone marrow. We have reported that B19 DNA, RNA and B19-VP protein may be detected on immunocytes in synovial membrane tissues of joints of patients suffering from rheumatoid arthritis¹⁰. Since it is thought that expression of P antigen is poor in these cells, there is a possibility that Ku80 plays an important role in the infection by B19 to immunocytes. It is expected that existence of a manner of infection by B19 through Ku80 not only contributes to the various symptoms of B19 infections diseases which were difficult to understand based on the relationship between the erythroblasts and B19, but also gives useful information for the diagnoses and therapies of B19 infectious diseases.
Reference 1: Plummer F A, Hammond G W, Forward K, Sekla L, Thompson L M, Jones S E, Kidd I M, Anderson M J: An erythema infectiosum-like illness caused by human parvovirus infection. N Engl. J. Med. 1985, 313: 74-9.
Reference 2: Anand, A., Gray, E. S., Brown, T., Clewley J. P., Cohen, B. J: Human parvovirus infection in pregnancy and hydrops fetalis. N Engl. J. Med. 1987, 316: 183-186.
Reference 3: Kelleher, J. F., Luban, N. L., Mortimer, P. P., Kamimura, T: Human serum “parvovirus”: a specific cause of aplastic crisis in children with hereditary spherocytosis. J. Pediatr. 1983, 102: 720-722.
Reference 4: White D G, Woolf A D, Mortimer P P, Cohen B J, Blake D R, Bacon P A: Human parvovirus arthropathy. Lancet 1985, 233: 419-21.
Reference 5: Reid D M, Reid T M, Brown T, Rennie J A, Eastmond C J. Human parvovirus-associated arthritis: a clinical and laboratory description. Lancet 1985, Feb 23: 422-5.
Reference 6: Brown, K. E., Anderson, S. M., & Young, N. S: Erythrocyte P antigen: cellular receptor for B19 parvovirus. Science 1993, 262: 114-117.
Reference 7: Brown, K. E. et al: Resistance to parvovirus B19 infection due to lack of virus receptor (erythrocyte P antigen). N Engl J. Med. 1994, 330: 1192-1196.
Reference 8: Barlow, G. D., & McKendrick, M. W. Parvovirus B19 causing leucopenia and neutropenia in a healthy adult. J. Infect. 2000, 40: 192-195.
Reference 9: Woolf, A. D., Campion, G. V., Chishick, A., Wise, S., Cohen, B. J., Klouda, P. T., Caul, O., Dieppe, P. A: Clinical manifestations of human parvovirus B19 in adults. Arch. Intern. Med. 1989, 149:1153-1156.
Reference 10: Takahashi, Y., Murai, C., Shibata, S., Munakata, Y., Ishii, T., Ishii, K., Saitoh, T., Sawai, T., Sugamura, K., Sasaki, T: Human parvovirus B19 as a causative agent for rheumatoid arthritis. Proc. Natl. Acad. Sci. USA 1998, 95: 8227-8232.
Reference 11: Stahl, H. D., Pfeiffer, R., von Salis-Soglio, G., Emmrich, F: Parvovirus B19-associated mono- and oligoarticular arthritis may evolve into a chronic inflammatory arthropathy fulfilling criteria for rheumatoid arthritis or spondylarthropathy. Clin. Rheumatol. 2000, 19: 510-511.
Reference 12: Weigel-Kelley, K. A., Yoder, M. C., & Srivastava, A: Recombinant human parvovirus B19 vectors: erythrocyte P antigen is necessary but not sufficient for successful transduction of human hematopoietic cells. J. Virol. 2001, 75: 4110-4116.
Reference 13: Doranz, B. J., Berson, J. F., Rucker, J., & Doms, R. W: Chemokine receptors as fusion cofactors for human immunodeficiency virus type 1 (HIV-1). Immunol Res. 1997, 16: 15-28.
Reference 14: Bergelson, J. M., Shepley, M. P., Chan, B. M., Hemler, M. E., & Finberg, R. W: Identification of the integrin VLA-2 as a receptor for echovirus 1. Science. 1992, 255: 1718-1720.
Reference 15: Summerford, C., Bartlett, J. S., & Samulski, R. J: AlphaVbeta5 integrin: a co-receptor for adeno-associated virus type 2 infection. Nat. Med. 1999, 5: 78-82.
Reference 16: Miyagawa, E., Yoshida, T., Takahashi, H., Yamaguchi, K., Nagano, T., Kiriyama, Y., Okochi, K., Sato, H: Infection of the erythroid cell line, KU812Ep6 with human parvovirus B19 and its application to titration of B19 infectivity. J. Virol. Methods 1999, 83: 45-54.
Reference 17: Yamaguchi K, Miyagawa E, Dan M, Miyazaki T, Ikeda H: Cellulose hollow fibers (BMMS) used in the filter membrane can trap human parvovirus (B19). Electron Microscopy 2002, 2: 115-116
Reference 18: Muryoi, T., Sasaki, T., Tamate, E., Takai, O., Harata, N., Yoshinaga, K: Antigen inhibition of the interaction between murine monoclonal anti-idiotypic antibodies and human monoclonal anti-DNA antibodies. Tohoku J. Exp. Med. 1987, 152: 253-258.
Reference 19: Mimori, T. et al: Isolation and characterization of cDNA encoding the 80-kDa subunit protein of the human autoantigen Ku (p70/p80) recognized by autoantibodies from patients with scleroderma-polymyositis overlap syndrome. Proc. Natl. Acad. Sci. USA 1990, 87: 1777-1781.
Reference 20: Tanaka, T. et al: Enhancement of antigen-induced T-cell proliferation by soluble CD26/dipeptidyl peptidase IV. Proc. Natl. Acad. Sci. USA 1994, 91: 3082-3086.
Reference 21: Brown, C. S., Salimans, M. M., Noteborn, M. H., & Weiland, H. T: Antigenic parvovirus B19 coat proteins VP1 and VP2 produced in large quantities in a baculovirus expression system. Virus Res. 1999, 15: 197-211.
Reference 22: Kajigaya, S. et al: Self-assembled B19 parvovirus capsids, produced in a baculovirus system, are antigenically and immunogenically similar to native virions. Proc. Natl. Acad. Sci. USA. 1991, 88: 4646-4650.
Reference 23: Nussenzweig, A. et al: Requirement for Ku80 in growth and immunoglobulin V(D)J recombination. Nature 1996, 382: 551-555.
Reference 24: Finnie, N. J., Gottlieb, T. M., Blunt, T., Jeggo, P. A., & Jackson, S. P: DNA-dependent protein kinase activity is absent in xrs-6 cells: implications for site-specific recombination and DNA double-strand break repair. Proc. Natl. Acad. Sci. USA 1995, 92: 320-324.
Reference 25: Lynch, E. M., Moreland, R. B., Ginis, I., Perrine, S. P., & Faller, D. V: Hypoxia-activated ligand HAL-1/13 is lupus autoantigen Ku80 and mediates lymphoid cell adhesion in vitro. Am. J. Physiol. Cell Physiol. 2001, 280: 897-911.
Reference 26: Ginis, I., & Faller, D. V: Hypoxia affects tumor cell invasiveness in vitro: the role of hypoxia-activated ligand HAL-1/13 (Ku86 autoantigen). Cancer Lett. 2000, 154: 163-174.
Reference 27: Teoh, G. et al: The 86-kD subunit of Ku autoantigen mediates homotypic and heterotypic adhesion of multiple myeloma cells. J. Clin. Invest. 1998, 101: 1379-1388.
Reference 28: Le Romancer M, Reyl-Desmars F, Cherifi Y, Pigeon C, Bottari S, Meyer O, Lewin M J: The 86-kDa subunit of autoantigen Ku is a somatostatin receptor regulating protein phosphatase-2A activity. J. Biol. Chem. 1994, 269: 17464-8.
Reference 29: Mostafa S S, Miller W M, Papoutsakis E T: Oxygen tension influences the differentiation, maturation and apoptosis of human megakaryocytes. Br. J. Haematol. 2000, 111: 879-89.
Reference 30: Hevehan D L, Papoutsakis E T, Miller W M: Physiologically significant effects of pH and oxygen tension on granulopoiesis. Exp. Hematol. 2000, 28: 267-75.
Reference 31: Sylvie Pillet, Nathalie Le Guyader, et al. Hypoxia up regulation the expression of human parvovirus B19. IX parvovirus workshop (2002 Italy)
Reference 32: Japanese Laid-open Patent Application (Kokai) No. 11-32757
Reference 33: Sakata H. et al. Vox Sang. 77(4), 197-B203, 1999)

We claim:

1. A method for binding human parvovirus B19 comprising:
bringing a receptor for human parvovirus B19 into contact with human parvovirus B19;
wherein said receptor consists essentially of a protein having the amino acid sequence shown in SEQ ID NO:1, or a protein having an amino acid sequence having a homology of not less than 90% with the amino acid sequence shown in SEQ ID NO:1.

2. A method for measuring human parvovirus B19 comprising the step of:
binding a receptor for human parvovirus B19 and human parvovirus B19;
wherein said receptor consists essentially of a protein having the amino acid sequence shown in SEQ ID NO:1, or a protein having an amino acid sequence having a homology of not less than 90% with the amino acid sequence shown in SEQ ID NO:1.

3. The method according to claim 2, comprising the steps of:
immobilizing said receptor or said protein on a solid phase;
bringing a sample containing B19 into contact with said immobilized receptor or protein;
reacting the resultant with an anti-B19 antibody or an antigen-binding fragment thereof, labeled with a marker after washing; and
measuring the marker bound to the solid phase after washing.

4. A method for adsorbing human parvovirus B19 comprising:
bringing human parvovirus B19 in a sample into contact with immobilized receptor for human parvovirus B19;
wherein said receptor consists essentially of a protein having the amino acid sequence shown in SEQ ID NO:1, or a protein having an amino acid sequence having a homology of not less than 90% with the amino acid sequence shown in SEQ ID NO:1.

5. The method according to claim 4, wherein said receptor or said protein is immobilized on a filter or a carrier packed in a column, and a sample containing human parvovirus B19 is passed through said filter or column.

6. The method according to claim 1, wherein said receptor consists essentially of a protein having a homology of not less than 95% with the amino acid sequence shown in SEQ ID NO:1.

7. The method according to claim 2, wherein said receptor consists essentially of a protein having a homology of not less than 95% with the amino acid sequence shown in SEQ ID NO:1.

8. The method according to claim 4, wherein said receptor consists essentially of a protein having a homology of not less than 95% with the amino acid sequence shown in SEQ ID NO:1.

9. The method according to claim 1, wherein said receptor consists essentially of a protein having a homology of not less than 90% with the amino acid sequence shown in SEQ ID NO:1, and wherein the amino acid substitutions are accomplished by substitution of an amino acid with an amino acid from the same Group, wherein the Groups consist of:
Group 1: Gly Ile, Val, Leu, Ala, Met, Pro;
Group 2: Asn, Gln, Thr, Ser, Tyr, Cys;
Group 3: Asp, Glu;
Group 4: Arg, Lys, His; and
Group 5: Phe, Tyr, Trp.

10. The method according to claim 2, wherein said receptor consists essentially of a protein having a homology of not less than 90% with the amino acid sequence shown in SEQ ID NO:1, and wherein the amino acid substitutions are accomplished by substitution of an amino acid with an amino acid from the same Group, wherein the Groups consist of:
Group 1: Gly Ile, Val, Leu, Ala, Met, Pro;
Group 2: Asn, Gln, Thr, Ser, Tyr, Cys;
Group 3: Asp, Glu;
Group 4: Arg, Lys, His; and
Group 5: Phe, Tyr, Trp.

11. The method according to claim 4, wherein said receptor consists essentially of a protein having a homology of not less than 90% with the amino acid sequence shown in SEQ ID NO:1, and wherein the amino acid substitutions are accomplished by substitution of an amino acid with an amino acid from the same Group, wherein the Groups consist of:
Group 1: Gly Ile, Val, Leu, Ala, Met, Pro;
Group 2: Asn, Gln, Thr, Ser, Tyr, Cys;
Group 3: Asp, Glu;
Group 4: Arg, Lys, His; and
Group 5: Phe, Tyr, Trp.

12. The method according to claim 1, wherein said receptor consists essentially of a protein having a homology of not less than 95% with the amino acid sequence shown in SEQ ID NO:1, and wherein the amino acid substitutions are accomplished by substitution of an amino acid with an amino acid from the same Group, wherein the Groups consist of:
Group 1: Gly Ile, Val, Leu, Ala, Met, Pro;
Group 2: Asn, Gln, Thr, Ser, Tyr, Cys;
Group 3: Asp, Glu;
Group 4: Arg, Lys, His; and
Group 5: Phe, Tyr, Trp.

13. The method according to claim 2, wherein said receptor consists essentially of a protein having a homology of not less than 95% with the amino acid sequence shown in SEQ ID NO:1, and wherein the amino acid substitutions are accomplished by substitution of an amino acid with an amino acid from the same Group, wherein the Groups consist of:
Group 1: Gly Ile, Val, Leu, Ala, Met, Pro;
Group 2: Asn, Gln, Thr, Ser, Tyr, Cys;
Group 3: Asp, Glu;
Group 4: Arg, Lys, His; and
Group 5: Phe, Tyr, Trp.

14. The method according to claim 4, wherein said receptor consists essentially of a protein having a homology of not less than 95% with the amino acid sequence shown in SEQ ID NO:1, and wherein the amino acid substitutions are accomplished by substitution of an amino acid with an amino acid from the same Group, wherein the Groups consist of:
Group 1: Gly Ile, Val, Leu, Ala, Met, Pro;
Group 2: Asn, Gln, Thr, Ser, Tyr, Cys;
Group 3: Asp, Glu;
Group 4: Arg, Lys, His; and
Group 5: Phe, Tyr, Trp.
